(19) United States
(12) Patent Application Publication
(10) Pub. No.: US 2006/0281139 A1
(43) Pub. Date: Dec. 14, 2006

(54) Carrier and method for the detection of anti-borrelia antibodies and test kit for use in the diagnosis of Lyme borreliosis infections

(75) Inventors: Martin Kintrup, Gauting (DE); Lilly Kronsteiner, Planegg (DE); Ludwig Furtmayr, Steingarden (DE); Vera Helbl, Munchen (DE)

Correspondence Address: MARSHALL, GERSTEIN & BORUN LLP, 233 S. WACKER DRIVE, SUITE 6300, SEARS TOWER, CHICAGO, IL, 60606, US

(73) Assignee: Viramed Biotech AG, Planegg/Steinkirchen (DE)

(21) Appl. No.: 11/438,199

(22) Filed: May 22, 2006

Related U.S. Application Data
(60) Provisional application No. 60/687,552, filed on Jun. 3, 2005.

(30) Foreign Application Priority Data
May 23, 2005 (EP) EP 05011134.3

(51) Int. Cl. G01N 33/554; C12M 1/34; C07K 14/195

(52) U.S. Cl. 435/7.32; 530/350; 435/287.2

(57) ABSTRACT

The invention relates to a carrier and a method for the detection of anti-Borrelia antibodies and a test kit for use in the diagnosis of Lyme borreliosis infections.

The invention relates to a cell-lysate-free carrier and a method for detecting anti-Borrelia antibodies, and a test kit for use in the diagnosis of Lyme borreliosis.

Lyme borreliosis is a common infectious disease in Europe with an annual incidence of an estimated 16 to 140 per 100,000 inhabitants. Lyme borreliosis is caused by the spirochete Borrelia burgdorferi, a spiral bacterium some 8 to 30 μm in length which can affect both humans and animals. A large number of possible interim hosts has been described. In Europe, the four genospecies B. afzelii, B. garinii, B. b. sensu stricto and B. valaisiana are transmitted most frequently through tick bites.

The progress of a typical Lyme borreliosis infection can generally be subdivided into three stages. The main symptom of Stage I is erythema (chronicum) migrans with a reddening of the skin which spreads over a period of days to weeks into a large round halo around the tick bite. Stage II is generally characterised by a neuroborreliosis, which can occur primarily as meningoradiculitis, meningitis or meningoencephalitis. Finally, in Stage III, Lyme arthritis, acrodermatitis chronica atrophicans or chronic-progressive borrelia encephalomyelitis are the most frequent manifestations.

However, the disease does not always run through all three stages; blurred transitions and symptom-free intervals are perfectly possible. Moreover, whole stages are frequently missed out, so that the disease can appear for the first time practically in any one of the three phases.

Because of the wide variety of clinical symptoms which are also very often similar to other forms of disease, and the fact that the medical history of the patient often provides no indication of a previous tick bite, it is necessary to supplement the diagnosis with laboratory study methods on a differential diagnostic basis. This is necessary only because, if the results are positive, a long period of treatment with antibiotics is indicated. One of the most significant factors in laboratory diagnostics is the detection of antibodies against Borrelia sp. (IgM, IgG) in serum, which can also be extended additionally to the patient's liquor in these of neurological manifestations of the disease.

On the principle of diagnosis in steps, a serological initial or search test for IgM and IgG antibodies is first carried out using enzyme-linked immunosorbent assay (ELISA) tests.

To restrict the antibody detection to anti-Borrelia antibodies, the search test is followed by what is called a confirmation test. This is generally a Western Blot test, which shows the antibody response of the patient to individual proteins of the bacterium in differentiated form. Using the strip pattern on the blot, in addition to detecting whether antibodies against Borrelia are present or not, information can also be obtained if required about the infection phase, since the immune response of the host differs significantly in the three stages of the infection in terms of prevalence and predominance of the antibodies of the various Ig classes. In the early phases, it is mainly IgM antibodies that are detected, whilst IgG antibodies prevail in the late phases.

In general, in the confirmation test during stage I of Lyme borreliosis, positive proof of antibodies in the serum can be expected in approx. 20 to 50% of the cases (WO 91/09870). Accordingly, in stage II, positive proof of antibodies can be expected in approx. 70 to 90% of cases, and in stage III, antibodies are almost always detected.

The state of the art already includes several confirmation tests which use either recombinant antigens derived from Borrelia burgdorferi sensu lato or gel electrophoretically separated whole cell lysates in Western Blot format.

DE 196 29 543, for example, describes a diagnostic agent for the detection of anti-Borrelia burgdorferi antibodies which is based on the recombinant antigens P870, P412, P402, P365, P315, P310, P280, P250, P230 and P130 derived from B. afzelii.

DE 198 47 142 describes how lysates from spirochete strains are separated using polyacrylamide SDS gel electrophoresis and, after transfer to nitrocellulose membrane, are brought into contact with an immune serum from a mouse. The antigens, which are used unpurified and are not expressed in in-vitro cultivation of Borrelia strains but only through in-vivo cultivation, have molecular masses in the polyacrylamide SDS gel of approx. 9.5, 18, 19, 30, 32, 33, 62, 70, 80, 90, 100 and 102 kDa.

The use of various recombinant antigens for serodiagnostic immunoblots based on p83/100, p39, OspC and p41 (flagellin) is described in Wilske et al., Med. Microbiol. Immunol. 1993, 182, 255-270 and Rössler et al., J. Clinic. Microbiol. 1997, 35, 2752-2758.

The published patent application DE 40 18 988 describes a Western Blot assay with various other proteins derived from Borrelia burgdorferi. The antigens used are recombinant and have molecular masses of 17, 22, 41 and 100 KDa.

An immunoblot for recombinant Osp17 and p58 is described in B. Wilske et al. Med. Microbiol. Immunol. 1999, 188, 139-144.

A further recombinant immunoblot which also contains VisE, DbpA and an OspC homologue is described by the same working group in U. Schulte-Spechtel et al., J. Clinic. Microbiol. 2003, 41, 1299-1303.

Similarly, WO 91/09870 describes how three recombinant antigens, p41 (recomb.), OspA (recomb.) and pC (recomb.) can be used in the ELISA method for detecting IgM antibodies and IgG antibodies against Borrelia in patients with erythema migrans.

What is disadvantageous in the confirmation tests in the state of the art is the fact that they detect a borreliosis infection in stage I of the disease in only 20 to 50% of cases (see WO 91/09870, for example). Because a complete clinical recovery is not often possible after a later stage of the disease has been reached, the beginnings of a Lyme borreliosis infection should, however, be diagnosed as early as possible.

The classification of Borrelia antigens on Western Blot strips using the full cell lysate is, moreover, dependent on the gel system used and can often only be evaluated by persons skilled in the art.

These tests vary considerably in terms of diagnostic value, and the user, i.e. the medical examination laboratory, and the requesting doctor are often unaware of this.

The state of the art confirmation tests which use gel electrophoretically separated whole cell lysates in the Western Blot format are sometimes very difficult to assess, since there are both specific and also non-specific bands (proteins) on the nitrocellulose strips, and, in addition, the bands (proteins) vary in their position on the carrier, rather than always being in the same position, because of the electrophoretic production process.

The assessment of a specific or non-specific reaction and the precise classification of the bands requires very experienced specialist staff and takes a good deal of time.

On the other hand, for state of the art confirmation tests with individually applied Borrelia proteins, to date only recombinant proteins have been available which, because of the lower antigenity, have a far lower sensitivity than the classic Western Blot. Diagnosis with these recombinant tests therefore provides results which are inadequate in terms of quality.

Both the tests using recombinant antigens and the tests with whole cell lysates in Western Blot format are often complicated in terms of execution/handling and for routine operation and are generally not suitable in particular for use in high throughput methods (HTS).

The object of the present invention is to provide a (diagnostic) carrier which is used for the detection of anti-Borrelia antibodies with a high degree of sensitivity and specificity and which combines the comprehensive specificity and sensitivity of the classic cell lysate Western Blot format with the user-friendliness and assessment reliability of tests with discretely applied individual Borrelia proteins—only available to date as recombinant proteins—and thus no longer demonstrates the above-mentioned shortcomings of existing tests.

Similarly, an object of the present invention is to provide a method for the detection of anti-Borrelia antibodies which is easy to standardise using wild-type Borrelia antigens. The antigens prepared with the method described have the advantage that their post-translational modifications correspond to those of the natural pathogens. This thus creates an advantage over recombinant antigens: because recombinant antigens are isolated from heterologous cells, they do not demonstrate any Borrelia-specific post-translational modifications. In order to reduce the toxicity of highly expressed membrane proteins, recombinant proteins often represent shortened derivates, which are frequently lacking epitopes compared with the wild-type proteins from the full cell lysates.

A position-specific application of the cleaned up antigens on a carrier produces a simpler evaluation and more reliable identification of the antigen-antibody complexes. In contrast to full cell lysates, the evaluation can be carried out safely and reliably by non-experts. The clear legibility of the test represents a clear advantage over the Western Blot method.

The object is furthermore to provide Borrelia-burgdorferi-specific antigens for the development of a diagnostic carrier for the detection of anti-Borrelia antibodies, whereby the antigens preferably: a) are isolated or extracted from homologous Borrelia strains, b) have a high antigenity with detectable antibodies in the early stage of the immune response and c) show the lowest possible cross-reactivity with antigens which are not specifically for Borrelia burgdorferi sensu lato.

In the development of a diagnostic test, particularly the choice of antigens which have a high degree of sensitivity and specificity for detection in the early stage of the infection is of crucial importance.

Subject matter of the invention is a cell-lysate-free carrier for the detection of anti-Borrelia antibodies, comprising at least two cleaned up wild-type antigens derived from Borrelia burgdorferi sensu lato (e.g. Borrelia burgdorferi sensu stricto, Borrelia afzelii, Borrelia garinii) which can bind to anti-Borrelia antibodies and are preferably present at different positions on the carrier separated from each other.

Antigens derived from Borrelia burgdorferi sensu lato (Borrelia antigens) are understood according to the invention to be preparations which contain diagnostically relevant antigens of Borrelia burgdorferi sensu lato. These are preferably antigens with a high degree of specificity for Borrelia burgdorferi sensu lato and a high reactivity in the various stages of Lyme borreliosis.

Particularly preferably, Borrelia antigens from Borrelia strains are used which show such a high antigen homology with the natural Borrelia strains occurring in the area of application of the test that borreliosis infections are detected with a high degree of sensitivity. For Germany, preferably, the strains B. afzelii, B. garinii and B. b. sensu stricto are suitable as an antigen source.

According to the invention, a wild-type antigen is an antigen expressed under homologous monitoring which corresponds to the natural antigen occurring in the cell. Wild-type proteins are understood in particular to be non-recombinant antigens which are obtained using a genetic engineering method, which is to say, for example, by infiltrating heterological DAN into the producer cell. In contrast to recombinant antigens, wild-type antigens have post-translational modifications. They occur either in a folded or in a completely or partly denaturised modification. Wild-type antigens which have been obtained from laboratory strains not occurring naturally are preferred.

Cleaning up Borrelia antigens with post-translational modifications from Borrelia cells produced, according to the invention, better reactivity than corresponding recombinant antigens and a comparable reactivity of the electrophoretically separated antigens in the Western Blot.

The Borrelia antigens are preferably partially denaturised and guarantee the optimum presentation of linear epitopes.

According to the invention, cleaned up means that the wild-type antigen is present in a purity of at least 50% by weight and preferably at least 90% by weight, and particularly preferably at least 95% by weight.

In an embodiment that is preferred above all, the purity is at least 99% by weight, whereby the residues consist solely of water and/or inorganic salts.

A “cell-lysate-free carrier” is a carrier which shows no component pattern that is typical for whole cell lysates. Such whole cell lysates contain a series of components which are non-specific and non-relevant to Borrelia, including proteins such as cellular proteins. These non-specific and non-relevant components do not include antigens derived from Borrelia burgdorferi sensu lato and are not completely contained in the “cell-lysate-free carrier” of the present invention. Consequently, the “cell-lysate-free carriers” of the present invention do not contain at least one such non-specific or non-relevant component which is typically contained in whole cell lysates. Preferably, the “cell-lysate-free” carrier is free from lipids and carbohydrates. More preferably, the “cell-lysate-free carrier” of the present invention does not contain any of the aforementioned non-specific and non-relevant components.

In a preferred embodiment, the carrier is made in such a way that the at least two wild-type antigens derived from Borrelia burgdorferi are chosen from the group consisting of 14 kD, 17 kD, 21 kD, 25 kD, 30 kD, 39 kD, 41 kD, 43 kD, 58 kD, 83 kD protein and DbpA.

In a preferred alternative embodiment, the at least two wild-type antigens derived from Borrelia burgdorferi are chosen from the group consisting of 18 kD, 25 kD (OspC), 28 kD, 30 kD, 31 kD, 39 kD, 41 kD, 45 kD, 58 kD, 66 kD, 83 (93) kD protein and DbpA.

In a particularly preferred embodiment, the carrier according to the invention comprises at least 2, or at least 4, or at least 6, or at least 8 or all different antigens chosen from the group consisting of 14 kD, 17 kD, 21 kD, 25 kD, 30 kD, 39 kD, 41 kD, 43 kD, 58 kD, 83 kD protein and DbpA or selected from the group consisting of 18 kD, 25 kD (OspC), 28 kD, 30 kD, 31 kD, 39 kD, 41 kD, 45 kD, 58 kD, 66 kD, 83 (93) kD protein and DbpA.

In a further embodiment, the carrier comprises additionally at least one further antigen which is recombinant and which is derived from Borrelia burgdorferi sensu lato.

The at least one further antigen can be according to the invention a complete or a truncated recombinant protein of Borrelia burgdorferi sensu lato. The recombinant antigens should preferably be well cleaned. In a preferred embodiment, the at least one recombinant antigen derived from Borrelia burgdorferi sensu lato is chosen from the group consisting of 14 kD, 17 kD, 18 kD, 21 kD, 25 kD (OspC), 28 kD, 30 kD, 31 kD, 39 kD, 41 kD, 43 kD, 45 kD, 58 kD, 66 kD and 83 kD (93) protein, VisE and DbpA.

In a particularly preferred embodiment of the test carrier, recombinant VisE is used as an additional test reagent.

In a preferred variant of the carrier, the at least one further recombinant antigen derived from Borrelia burgdorferi is a cleaned up antigen. According to the invention, cleaned up means that the antigen is present in a purity of at least 50% by weight and preferably in a purity of at least 90% by weight, and particularly preferably at least 95% by weight.

In a particularly preferred embodiment, the at least one further recombinant antigen derived from Borrelia burgdorferi is present in a purity of at least 99% by weight. It is preferred according to the invention that the residues of the antigen consist solely of water and/or inorganic salts.

In an alternative embodiment, the carrier comprises at least 3, or at least 4, or at least 6, or at least 8, or at least 10 or at least 12 different cleaned wild-type antigens derived from Borrelia burgdorferi sensu lato.

According to the invention, at least 2, or at least 3, or at least 4, or at least 6, or at least 8, or at least 10, or at least 12 or all of the cleaned wild-type antigens derived from Borrelia burgdorferi sensu lato should be in different concentrations on the carrier.

Every individual wild-type antigen can be modified independently to the guidelines of the national reference centres or titrated. With this type of embodiment of the carrier according to the invention, it is advantageous that overloading or underloading with the wild-type proteins can be excluded. This is often the case if Western Blot systems with electrophoretically separated whole cell lysates are used, since no independent titration or loading of the wild-type proteins can be undertaken. A recombinant Western Blot can be titrated in principle, but no defined positioning or local resolution takes place.

Through the defined application of cleaned wild-type antigens and the abandonment of electrophoresis, a clear decision can be made as to which antigen is reacting on the carrier.

According to the invention, the carrier comprises an antigen in a concentration if the antigen is present on the carrier with at least one antigen molecule. However, it is preferred that at least one antigen is present on the carrier in a concentration and quantity which can be detected by analytical methods known to the person skilled in the art and which is suitable for antibody diagnosis.

According to the invention, the carrier comprises additionally at least one control substance. This is suitable for notifying the proper execution of the method according to the invention or for providing data which are helpful in the evaluation of test results.

In a preferred embodiment, the at least one, control substance is a conjugate control substance independent of Borrelia, preferably selected from the group consisting if IgA, IgG and IgM conjugate control substances.

According to the invention, the control substance can be a cutoff control which is applied to the carrier or used independently as a biological material.

A band with a weaker signal than such a cutoff control is not evaluated, whilst an equally strong or stronger signal produces a valid band. The cutoff control can be, for example, a dilution of purified human, immunoglobulin or alkaline phosphatase. The cutoff control band is designed as a limit value indicator which indicates from which signal intensity the test result should be evaluated as positive or negative or, if necessary, questionable.

In a preferred embodiment, the diagnostic carrier comprises the following serum controls: a) one or more negative control serums, b) at least one positive control serum for the recognition of at least one and preferably all the diagnostically relevant bands, c) at least one cutoff control serum for the recognition of the limit-value area of the band intensity, whereby a band according to the invention is rated, as reactive if its intensity corresponds at least to that of the cutoff control.

Borrelia bands, according to the invention, are understood to be diagnostically relevant reactive and/or recognisable bands after the test has been carried out with a reactive sample. According to the invention, Borrelia bands are also understood to be reactive dots or lines if the carrier is structured in the dot-blot or line-blot format.

In a preferred embodiment, the carrier comprises a contact control which allows a statement to be made as to whether the carrier has come into contact with a biological sample to be tested. Because the biological sample to be tested is preferably an aqueous sample, i.e. contains water as the main component (i.e. 10 to 99% by weight) or secondary component (i.e. 1 to 10% by weight), the contact control in one embodiment is made as a moisture control. Corresponding substances which can be used as a contact control are known to the person skilled in the art. For example, dehydrated copper(II) sulphate or cobalt chloride can be used as simple substances to detect moisture with an aqueous biological sample.

In a particularly preferred embodiment, the contact control is made as a serum control. In this way, if the biological sample is serum, the fact that the carrier has been in contact with the biological sample can be proven. A possible serum control is, in particular, protein A or a corresponding other serum-binding substance.

In an advantageous embodiment, the carrier is made in such a way that it is produced as a fixed carrier on which the antigens are immobilised. The fixed carrier should preferably be selected from plate, strip, membrane, filter, paper, film or pearl.

In a particularly preferred embodiment, the carrier is designed as a test strip, in particular in a format which allows use in traditional immunodiagnostic methods. If cleaned wild-type antigens and/or cleaned recombinant or genetically engineered antigens are used as the reagent components in the diagnostic test, the antigens present in isolation are immobilised directly on the fixed carrier, preferably directly on the plates, strips, membranes, filters, papers, film or pearls. Microtitre plates may particularly be considered as the plates. The pearls can also be small spheres made from different materials on which the antigens are applied or immobilised.

In a further preferred embodiment, the carrier is designed as a Western Blot. The carrier comprises the reagent components in immobilised form.

In principle, all materials are suitable for producing the carrier which are known to persons skilled in the art as being used for the immobilisation of antigens.

In a particularly preferred embodiment, the carrier, however, consists of material chosen from the group of nitrocellulose, polyvinylidene difluoride (PVDF), polyamide, cellulose acetate and polystyrene. In the event that polyamide is used as the material, nylon is the preferred choice. Nitrocellulose, however, is, because of its physical surface properties, the material to be preferred above all others for the immobilisation of the antigens. In particular, it has been found that when nitrocellulose is used, a particularly good signal-to-noise ratio can be achieved for all diagnostically relevant band signals.

Moreover, the carrier can be a coated carrier. The coated part of the carrier then consists preferably of a firm material with such properties as are suitable for a coating. These are known to the person skilled in the art. The coating of the carrier comprises preferably a material selected from the above group of listed materials. Preferably, the coated part and the coating are joined to each other in such a way that the two parts cannot be accidentally separated.

In an alternative embodiment, however, a coated carrier can also be used on which the coating is joined to the coated part so that it can be removed if, for example, the coating is to be used for further analysis in a standardised method.

According to the invention, the antigens used can be applied directly to the coated carrier or applied together with the coating material onto the part to be coated.

In a preferred embodiment, the carrier is made with the help of a stripe technique which applies the cleaned antigens to particular positions and thus ensures the precise classification of the bands. The position-specific application of the cleaned antigens onto the carrier allows the antigen-antibody complexes to be evaluated easily and identified reliably. In this way, the evaluation can also be carried out safely and reliably by non-experts, in contrast to full cell lysates.

The ease with which the test is read out represents a clear advantage over the Western Blot system, since a use within HTS screening methods with automated operation is possible.

According to the invention, the carrier can be used as described above in the diagnosis of Lyme borreliosis infections or to monitor the progress of a Lyme borreliosis infection.

Subject matter of the present invention is a method for detecting anti-Borrelia antibodies. This method comprises the following steps according to the invention: 1) Provision of a carrier as described above, 2) incubation of a biological sample which may possibly contain anti-Borrelia antibodies with the carrier under conditions which allow the formation of at least one antigen-antibody complex, and 3) determination of the presence of anti-Borrelia antibodies in the sample through the detection of at least one antigen-antibody complex.

The method is also suitable as a means for diagnosing and/or monitoring the progress of a borreliosis infection. For this, the biological sample is a patient sample, that is to say, a sample which was taken from a human or animal body or a sample taken from a human or animal body and further processed.

According to the invention, the method or the carrier can detect antibodies against various genospecies of Borrelia burgdorferi sensu lato. The antibodies detected comprise, but are not limited to, antibodies against the following species: B. andersonii, B. afzelii, B. b. sensu stricto, B. garinii, Borrelia Group DN127, Borrelia Group DNPotiB2, Borrelia Group VS116, B. japonica, B. valaisiana. Preferably, the method or the carrier is used for the detection of anti-Borrelia antibodies against strains which have been proved to be pathogenic to humans, especially strains of the species, B. afzelii, B. garinii, B. b. sensu stricto, or strains which are under discussion as being pathogenic to humans, such as B. valaisiana.

If control substances are contained on the carrier, these can be used to indicate that the method according to the invention has been carried out properly. For example, a contact control indicates whether the carrier has been in contact with the biological sample for long enough. Similarly, a serum control shows whether, during the procedure, the carrier has contact with a serum. According to the invention, the control substances also allow conclusions to be drawn about whether the incubation of the biological sample with the carrier was long enough for the formation of at least one antibody-antigen complex. In this case, the control substance is an incubation time control or cutoff control.

Preferably, the detection method for anti-Borrelia antibodies is designed in such a way that it meets all the requirements as regards internal and external quality assurance under German standards DIN 58967-40 and DIN 58967-10. It is particularly preferred that the detection method additionally complies with the standard MIQ 2000 and US standard CDC.

In a preferred embodiment, the method is designed in such a way that at least one antigen reacts specifically with one anti-Borrelia antibody.

The biological sample for use in the method can be derived from an animal or a person. It is particularly preferred that it is a sample from a person.

Preferably, the biological sample is a liquid sample. In this case, blood, plasma, serum, urine, saliva, liquor or synovial fluid can be considered. It is particularly preferred that the biological sample is human blood, serum or Liquor cerebrospinalis. The biological sample can also be a further processed biological sample. Preferably, the further processing is such that the diagnostically relevant anti-Borrelia antibodies are concentrated in comparison with the original sample or are available in a higher degree of purity. Methods for the further processing of biological samples are known to the persons skilled in the art working in this field.

According to the invention, the biological sample can also be a liquefied sample which has been obtained through a further processing method from a tissue sample or a bioptate, preferably skin bioptate, joint bioptate or synovial bioptate.

For the detection of an antigen-antibody complex, in the diagnosis of Lyme borreliosis infections, the test kit according to the invention is used, which can also be used to monitor the progress of a Lyme borreliosis infection. In contrast to the detection method using PCR, this can produce a clear proof of an antigen-antibody complex. Such a test kit contains, in addition to the carrier, at least a substance for the detection of an antigen-antibody complex and/or at least a device for the detection of an antigen-antibody complex.

As part of the stepped diagnosis system for the diagnosis of Lyme borreliosis, it is possible, after a prior search test (e.g. ELISA, immuno-fluorescence test), to carry out both the serological confirmation reaction and also the serological progress monitoring using the carrier, method and test kit according to the invention. The test data provide a partial aspect for the decision regarding the need for treatment by the doctor in charge.

In a preferred embodiment, the test kit furthermore contains instructions for the evaluation of the test results which are obtained with the test kit. These instructions are intended to make it possible for the user to draw conclusions about the presence of anti-Borrelia antibodies in the biological sample. The instructions should preferably be set out in such a way that rules or criteria are laid down which make it possible to state whether the presence of anti-Borrelia antibodies 1) is positive, if anti-Borrelia antibodies are present, or 2) is negative, if anti-Borrelia antibodies are essentially not present, or 3) is questionable, if no statement can be made regarding the presence of anti-Borrelia antibodies.

In another embodiment, the instructions accordingly contain rules and criteria as to how information on concentrations of anti-Borrelia antibodies in the biological sample can be obtained.

In yet another embodiment, the carrier together with the instructions allow additional conclusions to be drawn as to which type of antibodies, preferably classified according to IgG, IgM or IgA antibodies, are present in the biological sample. By particular preference, the instructions also allow conclusions to be drawn as to the concentrations of the various antibodies.

In a further embodiment, the instructions contain information about how the test kit can be used to find out whether a test person from whom the biological sample to be examined has been taken is infected with Borrelia burgdorferi pathogens.

In yet another alternative embodiment, the instructions contain information as to how the test kit can be used for monitoring the progress of a Lyme borreliosis infection. In particular, the instructions contain rules or criteria of how the data on the disease pattern can be correlated. Preferably, a rough division into the three phases of the Lyme borreliosis infection will be possible here. The information is then based, according to the invention, on drawing a conclusion, by means of the presence of IgG, IgM and/or IgA antibodies, as the case may be, and if present preferably also the concentrations of the relevant antibodies, about the stage which a test person with Lyme borreliosis infection has reached.

Similarly, the instructions can contain information as to how the test kit can be used over a longer period of time to follow up a Lyme borreliosis infection.

The following embodiments are intended to explain the present invention in more detail without, however, having a limiting effect.

### EXAMPLE 1

Preparation of Membrane Fractions

All the steps were carried out at 4° C. Borrelia burgdorferi sensu lato cells were sedimented by 20 min centrifuging at 7000 g. The pellet was washed twice with cooled PBS buffer and the damp weight was determined. The washed cells were suspended in 10 ml cooled PBS buffer per g damp weight and disrupted by 2×10 min sonification (Branson sonifier). Through 60 min centrifugation at 140 000 g, soluble proteins were obtained in the supernatant and thrown away. The pellet was suspended in PBS twice as described above, sonified and centrifuged. The washed pellet contains the membrane fraction and further insoluble cell components.

### EXAMPLE 2

Isolation of Membrane Proteins Using Preparative Gel Electrophoresis

The membrane fraction was suspended in SDS polyacrylamide gel electrophoresis (SDS-PAGE) sample buffer (60 mM Tris Ci, pH 6.8, 2% SDS, 10% glycerine, 0.025% bromophenol blue and 5% mercaptoethanol) and dissolved by incubation at 100° C., so that the protein concentration was about 10 mg/ml. The solubilised proteins were fractionated through continuous elution SDS gel electrophoresis in a “PrepCell” (Model 491, Bio-Rad) in a gel system according to Laemmli (Laemmli, U K (1970), Nature 227: 680-5). Depending on the apparent molecular mass of the antigen to be isolated, a 6 cm high 7 to 15% separating gel and a 2 cm high 5% collector gel were used in a gel tube with an internal diameter of 37 mm. The proteins were separated for 14 to 20 h at a constant power of 12 W and eluated with an elution buffer (25 mM Tris CI, pH 8.3, 0.1% SDS) with a flow rate of 90 ml/h. Immediately after elution of the bromophenol blue front, the eluate was collected in 3 ml fractions. The UV absorption of the eluate did not provide any separated peaks which could be used for identification of the individual proteins. For this reason, the individual fractions were examined by analytical SDS-PAGE and Western Blot. The amount of protein was not sufficient in all cases for detection on the membrane by Ponceau S or in the gel using Coomassie Brilliant Blue. Using suitable mono- or oligo-specific serums, the target antigens, however, could be detected immunologically in the corresponding fractions (FIG. 1).

### EXAMPLE 3

Buffer Exchange and Concentrating the Extracted Borrelia Antigens

The fractions selected as suitable after analysis of the PrepCell runs were aggregated, buffer exchanged and concentrated. For this, concentrators made by Vivascience AG (Hamburg, Germany) were used according to the manufacturer's instructions (Vivaspin 15, no. VS15RH1, 5 kDa membrane). The solution from the aggregated fractions was applied to the concentrator in 15 ml aliquots and centrifuged for 20 mins each time at 5000 g and room temperature in a swing-out rotor. The retentate, which had been concentrated to around 10 times the original, was diluted with PBS 10× and centrifuged again. This washing stage was repeated 2 to 4 times. Finally, the retentate was concentrated to the point that a concentration of the antigen solution of 30 to 60× the initial volume was obtained. The solution was stored at −70° C. until further use.

### EXAMPLE 4

Application of the Concentrated Borrelia Antigen Solutions to the Carrier Nitrocellulose

The finished Borrelia antigen solutions were applied via a stripe process to the carrier nitrocellulose (Schleicher & Schuell, Dassel, Germany). For this, dispensing devices made by Biodot Ltd. (Huntingdon, GB) were used according to the manufacturer's instructions. The stripe conditions were chosen in such a way that the antigen solutions were applied as a homogenous line to the carrier. During the stripe process, the air humidity was 40-60% and the temperature was 19-23° C. After this, the membranes were blocked as standard according to the manufacturer's instructions.

The transfer of the proteins to the solid phase is carried out electrophoretically or by direct application to the immobilising matrix using a hole or slit mask (dot blot or line blot).

### EXAMPLE 5

Interpretation Criteria for a Positive Result

To evaluate the test results, the following interpretation criteria must be used, according to the invention. The criteria meet the requirements of the draft for the new German industrial standard DIN 58969-44.

The IgG blot should be evaluated as positive if at least two of the following bands are positive: 83 kD, 58 kD, 43 kD, 39 kD, 30 kD, 25 kD, 21 kD, 17 kD, 14 kD protein band, VisE band. The IgM blot is positive if at least one of the following bands is present: 41 kD (very marked), 39 kD, 25 kD, 17 kD protein bands.

If recombinant antigens were used for the IgG blot, the IgG blot is positive if at least two of the following bands are positive: 83 kD, 58 kD, 39 kD, 30 kD, 25 kD, 21 kD, 17 kD protein band, VisE, p41int band. The IgM blot is to be evaluated as positive if at least two of the following bands are present: 39 kD, 25 kD, 17 kD protein band, VisE, p41 int band. The IgM blot is also positive if the 25 kD band alone is present in marked form.

The test result is questionable if only one band appears in the immunoblot (apart from the 25 kD protein band in the IgM blot if the 25 kD protein is recombinant).

If no bands in the immunoblot are dyed, the result should be evaluated as negative.

### EXAMPLE 6

Performance Feature Data of the Carrier on IgG

Sensitivity:

From 68 clinically defined borreliosis-positive serums, the sensitivity of the diagnostic carrier was determined. For the early phase of the borreliosis infection, patient serums with erythema migrans, erythema chronicum migrans and multiple erythema migrantia were examined.

Of the patient serums in borreliosis stage I with erythema migrans (n=29), 9 (31%) were positive in the IgG blot, and 23 (79%) in the IgM/IgG blot. In borreliosis stage I with erythema chronicum migrans (n=27), 12 (44%) were positive in the IgG blot and 21 (78%) in the IgM/IgG blot. Of the serums in borreliosis stage I with multiple erythema migrantia (n=13), 9 (69%) were positive in the IgG blot and 10 (91%) in the IgM IgG blot.

Specificity:

To determine the specificity of the diagnostic carrier, 143 blood donor serums from South Germany were examined. A specificity of 140 (98%) was determined.

### EXAMPLE 7

Performance Feature Data of the Carrier on IgM

Sensitivity:

From 67 clinically defined borreliosis-positive serums, the sensitivity of the diagnostic carrier was determined. For the early phase of the borreliosis infection, patient serums with, erythema migrans, erythema chronicum migrans and multiple erythema migrantia were examined.

Of the patient serums in borreliosis stage I with erythema migrans (n=29), 20 (69%) were positive in the IgM blot, and 23 (79%) in the IgM/IgG blot. Of the serums in borreliosis stage I with erythema chronicum migrans (n=27), 16 (59%) were positive in the IgG blot and 21 (78%) in the IgM/IgG blot. Of the serums in borreliosis stage I with multiple erythema migrantia (n=13), 11 (85%) were positive in the IgG blot and 12 (92%) in the IgM/IgG blot.

Specificity:

To determine the specificity of the diagnostic carrier, 140 blood donor serums from South Germany were examined. A specificity of 132 (94%) was determined.

### KEY TO THE FIGURES

FIG. 1: Immunological identification of PrepCell, fractions by analytical Western Blot.

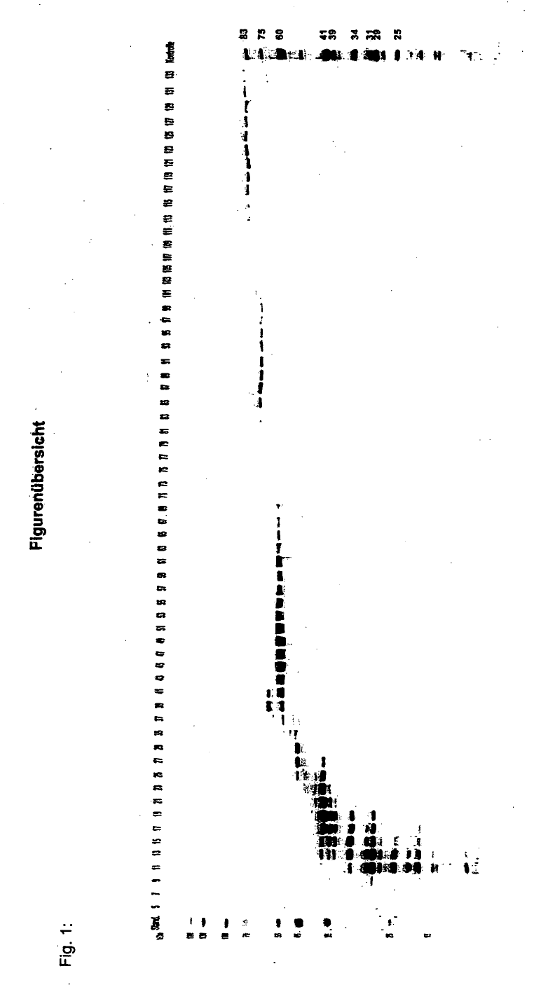

FIG. 2: Carrier according to the invention with anti-Borrelia antigens in various concentrations and applied at different points on the carrier, which also comprises VisE and DbpA, a serum control and conjugate controls (IgG, IgM and IgA).

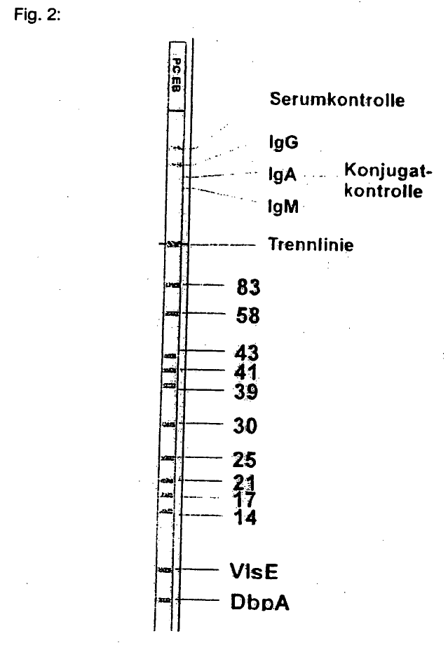

FIG. 3: Use of the carrier in an automated read and evaluation method for use in HTS diagnostics.

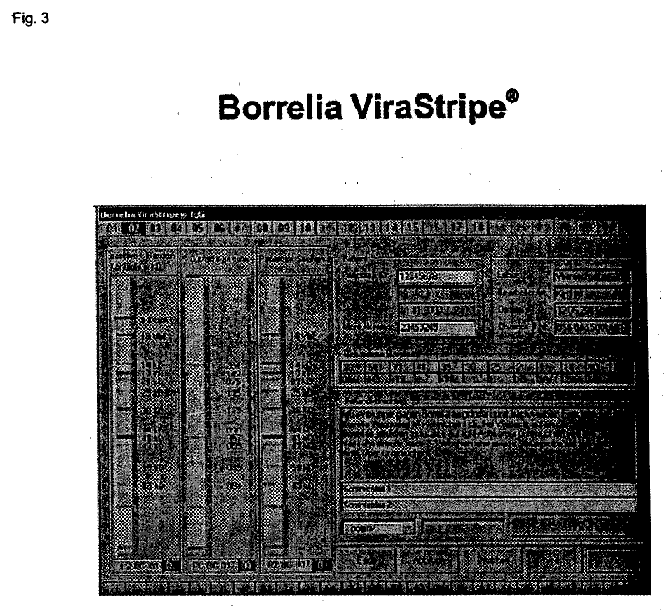

1. Cell-lysate-free carrier for the detection of anti-Borrelia antibodies, comprising at least two cleaned wild-type antigens derived from Borrelia burgdorferi sensu lato, which can bind to anti-Borrelia antibodies and which are preferably present on different carrier positions separated from each other.

2. Carrier according to claim 1, characterised in that the at least two cleaned wild-type antigens derived from Borrelia burgdorferi sensu lato are selected from the group consisting of 14 kD, 17 kD, 21 kD, 25 kD, 30 kD, 39 kD, 41′ kD, 43 kD, 58 kD, 83 kD protein and DbpA.

3. Carrier according to claim 1, characterised in that the at least two cleaned wild-type antigens derived from Borrelia burgdorferi sensu lato are selected from the group consisting of 18 kD, 25 kD (OspC), 28 kD, 30 kD, 31 kD, 39 kD, 41 kD, 45 kD, 58 kD, 66 kD, 83 (93) kD protein and DbpA.

4. Carrier according to one of claims 1 to 3, characterised in that this comprises additionally at least one recombinant antigen derived from Borrelia burgdorferi sensu lato.

5. Carrier according to claim 4, characterised in that the at least one recombinant antigen derived from Borrelia burgdorferi sensu lato is selected from the group consisting of 14 kD, 17 kD, 18 kD, 21 kD, 25 kD (OspC), 28 kD, 30 kD, 31 kD, 39 kD, 41 kD, 43 kD, 45 kD, 58 kD, 66 kD and 83 kD (93) protein, VisE and DbpA.

6. Carrier according to claim 4, characterised in that the at least one recombinant antigen derived from Borrelia burgdorferi sensu lato is VisE.

7. Carrier according to one of claims 4 to 6, characterised in that the at least one recombinant antigen derived from Borrelia burgdorferi sensu lato is a cleaned antigen.

8. Carrier according to one of claims 1 to 7, characterised in that this comprises at least 3, or at least 4, or at least 6, or at least 8, or at least 10, or at least 12 different cleaned wild-type antigens derived from Borrelia burgdorferi sensu lato.

9. Carrier according to one of claims 1 to 8, characterised in that at least 2, or at least 3, or at least 4, or at least 6, or at least 8, or at least 10, or at least 12, or all of the cleaned wild-type antigens derived from Borrelia burgdorferi sensu lato are present on the carrier in different concentrations.

10. Carrier according to one of the claims 1 to 9, characterised in that it comprises additionally at least one control substance.

11. Carrier according to claim 10, characterised in that the at least one control substance is a Borrelia-independent conjugate control substance, preferably selected from the group consisting of IgA, IgG and IgM conjugate control substances.

12. Carrier according to claim 10 or 11, characterised in that the carrier comprises at least one contact control which allows a statement to be made as to whether the carrier has been in contact with a biological sample.

13. Carrier according to one of the claims 1 to 12, characterised in that the carrier is a fixed carrier on which the antigens are immobilised, wherein the fixed carrier is preferably selected from plate, strip, membrane, filter, paper, film or pearl.

14. Carrier according to claim 13, characterised in that the fixed carrier is selected from a material from the group comprising nitrocellulose, polyvinylidene difluoride (PVDF), a polyamide, nylon, cellulose acetate and polystyrene.

15. Carrier according to one of the claims 1 to 14 for use in the diagnosis of Lyme borreliosis infection or for monitoring the progress of a Lyme borreliosis infection.

16. Method for the detection of anti-Borrelia antibodies comprising the following steps:
a) Provision of a carrier according to one of the claims 1 to 15;
b) Incubation of a biological sample which may contain anti-Borrelia antibodies with the carrier under conditions which allow the formation of at least one antibody-antigen complex; and
c) Determination of the presence of anti-Borrelia antibodies in the biological sample through the detection of at least one antigen-antibody complex.

17. Method according to claim 16, characterised in that at least one antigen reacts specifically with an anti-Borrelia antibody.

18. Method according to one of the claims 16 or 17, characterised in that the biological sample has been derived from an animal or a human.

19. Method according to claim 18, characterised in that the biological sample derived from an animal or human is a liquid sample, preferably blood, plasma, serum, urine, liquor, saliva, liquefied tissue sample or synovial fluid.

20. Test kit for use in the diagnosis of Lyme borreliosis infections or for monitoring the progress of a Lyme borreliosis infection comprising the carrier according to one of the claims 1 to 15 and additionally at least one substance for the detection of an antigen-antibody complex.

21. Test kit according to claim 20, characterised in that the test kit also contains instructions for the evaluation of the test results obtained.
